(19) United States
(12) Patent Application Publication
(10) Pub. No.: US 2026/0367090 A1
(43) Pub. Date: Nov. 19, 2026

(54) ANTIBODY DIMERIZATION DETECTED BY MULTIFACETED MASS SPECTROMETRY

(71) Applicant: Regeneron Pharmaceuticals, Inc., Tarrytown, NY (US)

(72) Inventors: Andrew J. Heindel, Tarrytown, NY (US); Shunhai Wang, Scarsdale, NY (US); Yuetian Yan, Chappaqua, NY (US)

(73) Assignee: Regeneron Pharmaceuticals, Inc., Tarrytown, NY (US)

(21) Appl. No.: 19/674,611

(22) Filed: May 12, 2026

Related U.S. Application Data
(60) Provisional application No. 63/806,271, filed on May 15, 2025.

(51) Int. Cl. G01N 33/68; B01D 15/34; G01N 30/06; G01N 30/72

(52) U.S. Cl. CPC G01N 33/6848; B01D 15/34; G01N 30/06; G01N 30/72

(57) ABSTRACT

Antibody cocktails are becoming an increasingly popular therapeutic class; however, antibody cocktails present analytical challenges over traditional single monoclonal antibody (mAb) formulations. One paramount concern is the formation of heteromeric species that have unknown impacts on safety and efficacy. Consequently, effective approaches for identifying and characterizing high-molecular weight (HMW) impurities are critical to the successful development of this therapeutic class. In this study, a multifaceted mass spectrometry approach was used to characterize a unique dimer species formed between two mAbs under thermal stress, revealing an intriguing dimerization mechanism that is driven by Complementarity-Determining Region (CDR)-clipping. Size exclusion chromatography-mass spectrometry (SEC-MS), complemented by post-column denaturation (PCD), was utilized at both intact and subunit levels to pinpoint the dimerization interface. These results highlight the critical role of sophisticated analytical methods in comprehending and addressing the complexities linked to mAb cocktails.

### CROSS-REFERENCE TO RELATED APPLICATIONS

This application claims the benefit of U.S. Provisional Application No. 63/806,271, filed May 15, 2025, the entire contents of which are expressly incorporated herein by reference.

### REFERENCE TO ELECTRONIC SEQUENCE LISTING

The application contains a Sequence Listing which has been submitted electronically in. XML format and is hereby incorporated by reference in its entirety. Said .XML copy, created on May 11, 2026, is named “135975-68002.xml” and is 3,782 bytes in size. The sequence listing contained in this .XML file is part of the specification and is hereby incorporated by reference herein in its entirety.

### FIELD

The inventions provide methods of characterizing aggregates in an antibody (mAb) mixture comprising more than one antibody species to facilitate improved development and production methodologies. Systems for performing the methods also are provided.

### BACKGROUND

Therapeutic antibodies have emerged as a crucial treatment option for various severe diseases, with over a hundred monoclonal antibody (mAb) products approved by regulatory agencies in the past 35 years [1,2]. This widespread adoption underscores their importance; however, despite their popularity, there are inherent limitations in using a single, highly specific molecule with a single target. Mutations in the antigen render single therapies ineffective [3] highlighted by COVID-19 [4] and cancer [5]. To combat mutation, the immune system relies on multiple mAbs targeting a host of different epitopes [6,7]. Consequently, pharmaceutical companies are employing various modalities and strategies to create more effective therapeutic interventions that more closely resemble a natural immune response. Examples of strategies to increase clinical efficacy include bispecific antibodies and combination products [8-10]. Combination products offer a variety of approaches for the concurrent administration of multiple therapeutics. These strategies include methods like dual-barrel syringes and multiple infusion bag single lines, which facilitate co-administration of existing formulations. Additionally, there are more integrated techniques such as co-formulation, where therapeutics are combined into a single product for simultaneous delivery.

Co-formulation represents the purist approach to combination therapy, where multiple drug substances are combined into a single formulation to deliver therapeutic agents concurrently, maximizing clinical efficacy and patient convenience. This method not only enhances clinical outcomes by harnessing synergistic effects but also simplifies treatment protocols, reducing administration frequency and minimizing the risk of medical errors. [8, 10]. As a result, numerous pharmaceutical companies are investigating co-formulation opportunities. Three fixed-dose combinations (FDCs) are approved in the United States as of 2022 [10-14]. There are also several other FDCs that are currently in various stages of clinical trials [10]. While co-formulation offers significant therapeutic advantages, it also introduces complex analytical challenges that must be addressed to ensure product safety and efficacy.

FDCs present unique analytical challenges due to the increased sample complexity, necessitating additional attention during their development [15-18]. Co-formulated antibodies, like their single-mAb formulation counterparts, can undergo changes in post-translational modifications (PTMs), charge and size variants under storage and stress conditions due to various physical, chemical or enzymatic degradations [16, 17, 19-22]. In particular, the high-molecular weight (HMW) species often pose significant analytical challenges due to their considerable heterogeneity in size, conformation, and interaction mechanisms [23]. Unlike individually formulated mAbs, co-formulated antibodies can potentially interact, forming heteromeric species. These HMW species have unknown impacts on product safety and efficacy [15, 16, 24, 25], necessitating effective techniques to monitor and characterize them throughout the product lifecycle.

Common techniques for elucidating HMW species in single-mAb formulations include sedimentation velocity analytical ultracentrifugation (SV-AUC) [26, 27], size-exclusion chromatography (SEC) coupled with ultraviolet detection (UV) [28, 29], and SEC-multiangle light scattering (MALS) [30]. However, co-formulated mAb products present elevated analytical challenges, as the mAbs are usually similar in size (i.e., hydrodynamic radii), making it nearly impossible to distinguish various dimer species by SEC. Coupling SEC with other techniques such as surface plasmon resonance (SPR) can provide detailed information on specific antibodies in aggregates. For example, aggregates obtained from forced degradation studies on a mixture of two mAbs were collected by SEC and analyzed by SPR, demonstrating that only one of the antibodies was present in the mixture [16]. However, utilizing SPR requires an assumption that the binding epitopes are accessible in the oligomer, which is not necessarily true depending on the geometry of the aggregate species. Another approach to dissect oligomer identity is SEC couple with mass spectrometry (SEC-MS) whether under denaturing conditions or near native conditions (nSEC-MS) [32]. Native SEC-MS preserves both covalent and non-covalent oligomeric interactions. In addition, very low levels of HMW impurities (0.01%) can be discerned when using state-of-the-art high-performance liquid chromatographs (HPLC) and high-resolution mass spectrometers [33]. While nSEC-MS preserves both covalent and non-covalent interactions, the technique is not able to distinguish between the two interaction types unless clear mass differences (e.g., from the crosslinks) are detected. Reduced mass accuracy is also experienced during mass deconvolution of HMW species due to their low abundances and relatively poor MS responses. Consequently, unconventional HMW species, which may emerge from co-formulated mAb products, are difficult to decipher. Post-column denaturation (PCD)-assisted SEC-MS (SEC-PCD-MS) alleviates many of these shortcomings by separating the low-abundance HMW species natively and subsequently breaking them into its monomeric constituents prior to MS detection [25]. This technique permits unequivocal identification of low levels of HMW species without requiring enrichment, which can be time-consuming and may introduce artifactual HMW species.

The inventions provide for, among other things, identification of a novel dimerization mechanism in a co-formulated mAb cocktail using a multifaceted MS-based approach. Using thermally stressed material, rapid growth of a unique truncated dimer species in a co-formulated mAb cocktail using nSEC-MS was observed. Following this observation, SEC-PCD-MS was performed at the intact, Fab2 and subunit levels to identify the truncation site and the domains responsible for dimerization. These analyses uncovered an intriguing dimerization mechanism that was driven by CDR-clipping of one mAb, followed by the two mAb dimerizing at the dimerization region (the domain opposite the clipped CDR) and a full length Fab arm. This mechanism has also been referred to as “domain swapping” between the two mAb molecules. Finally, by probing the disulfide bond susceptibility changes via limited reduction and middle-down analysis, the structural changes of the involved domains were studied. These findings underscore the importance of advanced analytical techniques in understanding and mitigating the challenges associated with co-formulated biotherapeutics.

### SUMMARY

The inventions provide methods of identifying a dimerization region in a first antibody in an antibody mixture, wherein the method comprises the steps of (a) subjecting the antibody mixture to stress; (b) conducting native size-exclusion chromatography (nSEC) on the stressed antibody mixture from step (a); (c) applying post-column denaturation (PCD) solvent after nSEC separation, thereby enabling dissociation of high-molecular weight (HMW) species into their monomeric constituent components; and (d) analyzing the PCD solvent treated components by mass spectrometry (MS), thereby identifying the dimerization region in the first antibody in the antibody mixture.

The antibody mixture of step (a) can be subjected to thermal stress. The stress may be selected from thermal stress, photo stress, low pH stress, agitation stress, sheer stress or combinations thereof, for example. In a particular embodiment, the stress is thermal stress. In a particular embodiment, the stress is thermal stress.

The inventions provide methods of identifying a dimerization region in a first antibody in an antibody mixture, wherein the method comprises the steps of (a) subjecting the antibody mixture to thermal stress; (b) conducting native size-exclusion chromatography (nSEC) on the thermal stressed antibody mixture from step (a); (c) applying post-column denaturation (PCD) solvent after nSEC separation, thereby enabling dissociation of high-molecular weight (HMW) species into their monomeric constituent components; and (d) analyzing the PCD solvent treated components by mass spectrometry (MS), thereby identifying the dimerization region in the first antibody in the antibody mixture.

The antibody mixture of step (a) can be heated to a temperature about 30 °C, 31 °C, 32 °C, 33 °C, 34 °C, 35 °C, 36 °C, 37 °C, 38 °C, 39 °C, 40 °C, 41 °C, 42 °C, 43 °C, 44 °C, 45 °C, 46 °C, 47 °C, 48 °C, 49 °C, or 50 °C.

The antibody mixture of step (a) can be heated to a temperature greater than 40 °C. The antibody mixture of step (a) can be heated to a temperature greater than 45 °C.

The antibody mixture of step (a) can be stressed for about 1 hour to about 6 months. The antibody mixture of step (a) can be thermally stressed for about 1 hour to about 6 months. The antibody mixture of step (a) can be stressed for about 1 month or about 3 months. The antibody mixture of step (a) can be thermally stressed for about 1 month or about 3 months.

The MS analysis of step (d) can further compare HMW species from the antibody mixture and HWM species from a single-antibody sample of the first antibody. The MS analysis of the dimerization region can reveal the presence of a truncated antibody species in both the antibody mixture and single-antibody sample of the first antibody.

The PCD treatment can reveal an increased level of a truncated antibody species and its complementary fragment. The truncated antibody species can be truncated at a complementarity-determining region (CDR). The truncated antibody species can include a domain opposite the truncated CDR. The domain opposite the truncated CDR can be the dimerization region.

The antibody mixture can include the first antibody, a second antibody, and optionally one or more additional antibodies.

An intact Fab2 arm of the second antibody can dimerize with the dimerization region.

The antibody mixture can include monoclonal antibodies.

The antibody mixture can be in Tris-HCl buffer having a pH ranging from 7.0 to 8.5.

The method can include digesting the antibody mixture with IdeS prior to nSEC in step (b) to separate a fragment crystallizable region (Fc) and a Fab2 arms.

The IdeS-treated antibody mixture can be subjected to steps (b)-(d).

The subsequent PCD-MS analysis can identify that the dimerization region includes a truncated Fab2 from the antibody and an intact Fab2 is from the second antibody, thereby identifying the dimerization region comprise a Fab-Fab interaction.

The IdeS-treated sample can be subjected to partial reduction under native conditions to selectively reduce the inter-chain disulfide bonds and further localize the interaction to the constituent subunits, thereby determining the domains involved in the dimerization region.

Systems for performing the above methods also are provided.

These systems can include systems for characterizing a dimerization region in an antibody mixture comprising a first antibody, wherein the system comprises a native size-exclusion chromatography (nSEC), a post-column denaturation (PCD) solvent, and mass spectrometry (MS) (SEC-PCD-MS) to analyze the antibody mixture to characterize at least one multimeric antibody form comprising one or more truncated antibody species and at least one multimeric antibody form comprising one or more full length antibody species in one or more elution peaks.

The multimeric antibody form can be selected from the group consisting of a dimer, trimer, tetramer, pentamer, hexamer, and heptamer forms.

The antibody mixture can be subjected to thermal stress.

The SEC-PCD-MS can generate extracted ion chromatograms to elucidate the truncated antibody species.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H schematically depict enrichment of truncated mAb in dimer region. SEC-UV data are shown of co-formulated (FIG. 1A) and single formulated (FIG. 1B) mAbs in thermally stressed samples, highlighting the dimer (orange, 15× zoom) and monomer region (blue). Percentages were calculated using UV area of dimer region and main peak. Deconvoluted mass data derived from dimer region of co-formulated (FIG. 1C) and single formulated (FIG. 1D) mAbs. Constituent parts of the dimers are displayed in blue (mAb-1, mAb-1T) and red (mAb-2). Deconvoluted mass data are derived from dimer region of co-formulated (FIG. 1E) and single formulated (FIG. 1F) mAbs after PCD. Deconvoluted mass data are derived from monomer region of co-formulated (FIG. 1G) and single formulated (FIG. 1H) mAbs after PCD. Percentages represent the area fraction of individual components relative to the area of full-length mAb-1. Red circles highlight the clipped species.

FIGS. 2A-2B schematically depict dimerization-induced VH loss in mAb-1: a mechanistic insight. (FIG. 2A) Deconvoluted mass spectrum of single mAb formulation obtained under native (left) or PCD (right) conditions are shown. (FIG. 2B) SEC-UV and extracted ion chromatograms (XICs) of mAb species from monomer region are shown. XICs were generated using a single charge state of each species.

FIG. 3 schematically depicts a treatment scheme digesting mAb products with IdeS.

FIGS. 4A-4B depict dimer Interactions Independent of mAb-2 LC. (FIG. 4A) Deconvoluted mass spectra of truncated dimer before (top) and after PCD (bottom). (FIG. 4B) nSEC-UV and XICs of mAb-1T and full-length mAb-2 domains after partial reduction and alkylation. XICs were generated using a single charge state of each species.

FIGS. 5A-5B depict Solvent Exposure of mAb-2 HC CH1 Domain Due to Loss of mAb-2 LC. (FIG. 5A; SEQ ID NO: 1) HCD MS2 fragmentation patterns for mAb-2 HC. The observed y-ion HCD fragments are shown in blue. The signature fragment ions used for XICs are highlighted in red. (FIG. 5B) Zoom-in view of the signature fragment ions are shown. Expected isotope envelopes are red, while observed are in green.

FIG. 6 schematically depicts ‘Triangular’ heterocomplex model formed by mAb-1 LC, mAb-1 FdT (VH-Framework 4, FW4 and CH1) and mAb-2 Fd (VH-CH1). Left: Cleaved mAb-1 Fab. The dashed box highlights the VH FW4 region following the cleavage. Right: mAb-2 Fd. The dashed box highlights CH1 from mAb-2. Potential rotations of highlighted regions needed to form the complex are indicated by the curved arrows. Middle: Structural model of possible heterocomplex formation between cleaved mAb-1 Fab and mAb-2 Fd. Hydrophobic patches are depicted with matching colors to their corresponding Fab chains. Cyan: mAb-1 LC; Blue: mAb-1 FdT; Red: mAb-2 Fd.

FIGS. 7A-7B show identification of full-length and truncated mAb 1 (mAb1*) peptide via reduced peptide mapping. (FIG. 7A) Extracted ion chromatograms (XICs) of full-length (top) and truncated (bottom) peptides are shown. (FIG. 7B; SEQ ID NOs: 2 and 3) HCD MS2 fragmentation patterns of full-length (top) and truncated (bottom) peptide from mAb1 are shown. The observed y-ion (red) and b-ion (blue) HCD fragments are displayed.

FIGS. 8A-8E show nSEC-MS revealing dimer interactions, which are dependent on Fab2 arms. (FIG. 8A) SEC-UV of co-formulated mAb sample (T=3M, 37 C) is shown. Deconvoluted masses of dimer (FIG. 8B) and monomer (FIG. 8C) regions post-IdeS digestion using nSEC-MS are shown. Deconvoluted mass of dimer (FIG. 8D) and monomer (FIG. 8E) regions after IdeS digestion using nSEC-MS-PCD are shown.

FIGS. 9A-9C show loss of mAb2 LC which leads to additional alkylation of mAb2 Fd in dimer region. (FIG. 9A) nSEC-UV and XICs of mAb2 components from co-formulated sample (T=3M, 37 C) are shown. Deconvoluted masses of dimer (FIG. 9B) and monomer (FIG. 9C) regions post-IdeS digestion, partial reduction and alkylation analyzed using nSEC-MS followed by PCD are shown.

FIG. 10 show raw MS2 spectra of the mAb2 fragment featuring two additional iodoacetamide modifications.

FIGS. 11A-11C show a fragmentation pattern of mAb2 Fd from monomer region highlights full complement of disulfide bonds. (FIG. 11A; SEQ ID No: 1) Raw MS2 spectra of the mAb2 fragment featuring traditional disulfide bonding are shown. (FIG. 11B) HCD MS2 fragmentation patterns for mAb2 Fd are shown. The observed b-ion and y-ion HCD fragments are shown in blue. The signature fragment ions used for XICs are highlighted in red (FIG. 11C) Zoom-in view of the signature fragment ions. Expected isotope envelopes are green, while observed (“found”) are in red.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

### ABBREVIATIONS

mAb: monoclonal antibody; truncated mAb-1: mAb-1T; FdT: truncated Fd; HMW: high molecular weight; CDR: Complementarity-Determining Region; PCD: post-column denaturation; SEC: size exclusion chromatography; MS: mass spectrometry; PTMs: post-translational modification; SV-AUC: Sedimentation Velocity Analytical Ultracentrifugation; UV: ultraviolet; MALS: multi-angle light scattering; SPR: surface plasmon resonance; FDC: fixed dose combination; HPLC: high-performance liquid chromatography; Da: Daltons; HC: heavy chain; VH: variable heavy; XIC: extracted ion chromatogram; FC: Fragment Crystallizable; MW: molecular weight; CH1: first constant heavy chain domain; LC: light chain; IAA: Iodoacetamide; HCD: Higher-Energy Collisional Dissociation; Fd: Fragment, heavy chain domain; FW4: Framework 4; FA: Formic Acid; ACN: Acetonitrile; TFA: Trifluoroacetic Acid; TCEP: Tris(2-carboxyethyl)phosphine; IPA: Isopropyl Alcohol; U: Units; mL: milli Liters; nESI: nano-Electrospray Ionization; UHMR: Ultra-High Mass Range; w/w: Weight/Weight.

### DEFINITIONS

Unless defined otherwise, all technical and scientific terms used herein have the same meaning as commonly understood by one of ordinary skill in the art to which this invention belongs. The term “about” in the context of numerical values and ranges refers to values or ranges that approximate or are close to the recited values or ranges such that the invention can perform as intended, such as having a desired number, value, rate, amount, density, degree, increase, decrease, percentage, ratio, value, purity, pH, concentration, presence of a form or variant, temperature or amount of time, as is apparent from the teachings contained herein. For example, “about” can signify values either above or below the stated value in a range of approximately +/−10% or more or less depending on the ability to perform. Thus, this term encompasses values beyond those simply resulting from systematic error.

Amounts and relative amounts of excipients, ingredients, and other materials may be described by mass, or moles. Units of mass may be expressed as grams, milligrams, micrograms, and the like). The term “weight” as in “weight/volume” or “w/v” means “mass”. Relative amounts may be expressed as percent weight (i.e., percent mass), wherein one (1) percent weight to volume (w/v) means 1 gram of material per 100 milliliter of volume. Also for example, one (1) part ingredient “A” per one (1) part ingredient “B” by weight means e.g., that for every one (1) gram of ingredient “A” there is one (1) gram of ingredient “B”. Also for example, one percent (1%) by weight of ingredient “A” means e.g., that for every 100 grams of total mass of a particle there is one (1) gram of ingredient “A”. Relative amounts of an ingredient may also be expressed in terms of moles or number of molecules per given volume, e.g., millimoles per liter (millimolar (mM)), or per other ingredient, e.g., X part ingredient “A” per Y part ingredient “B” by mole means for every X moles of “A” there are Y moles of “B”.

Although any methods and materials similar or equivalent to those described herein can be used in the practice or testing of the present invention, the preferred methods and materials are now described. All publications mentioned herein are incorporated herein by reference to describe in their entirety.

### DESCRIPTION

Post-column denaturation-assisted nSEC-MS (SEC-PCD-MS) method disassociates SEC-resolved, non-covalent HMW species into constituent components for subsequent MS detection. As a result, non-covalent and non-dissociable HMW species under identical SEC separation conditions are simultaneously detected. Identification of heterogeneous HMW species is improved by 1) confirming the identities of the constituent subunits dissociated from the non-covalent HMW complexes; and 2) achieving more accurate mass measurement of non-dissociable HMW species by removing interference from co-eluting, non-covalent species. Furthermore, incorporating a limited enzymatic digestion step (for example, IdeS digestion) both the interaction nature and interaction interfaces of mAb aggregates at subdomain levels were readily revealed. See International Publication No. WO2023043733A1.

Post-column denaturation-assisted SEC-MS method (SEC-PCD-MS) allows rapid and in-depth characterization of mAb aggregates directly from drug samples, and can also characterize mAb aggregates in samples containing more than one antibody species. This technique was used to re-examine the impact of multiple antibody species in an antibody mixture on the overall stability of mAbs in the mixture and when under thermal stress conditions encountered in forced degradation studies.

The term “protein” means any amino acid polymer having more than about 50 amino acids covalently linked via amide bonds. Proteins contain one or more amino acid polymer chains, generally known in the art as “polypeptides”. A protein may contain one or multiple polypeptides to form a single functioning biomolecule. “Polypeptides” generally contain over 50 amino acids, whereas “peptides” generally contain 50 amino acids or less.

Proteins may contain various covalent and non-covalent modifications. Disulfide bridges (i.e., between cysteine residues to form cystine) may be present in some proteins. These covalent links may be within a single polypeptide chain, or between two individual polypeptide chains. For example, disulfide bridges are essential to proper structure and function of insulin, immunoglobulins, protamine, and the like. For a recent review of disulfide bond formation, see Oka and Bulleid, “Forming disulfides in the endoplasmic reticulum,” 1833(11) Biochim Biophys Acta 2425-9 (2013).

In addition to disulfide bond formation, proteins may be subject to other post-translational modifications. Those modifications include lipidation (e.g., myristoylation, palmitoylation, farnesoylation, geranylgeranylation, and glycosylphosphatidylinositol (GPI) anchor formation), alkylation (e.g., methylation), acylation, amidation, glycosylation (e.g., addition of glycosyl groups at arginine, asparagine, cysteine, hydroxylysine, serine, threonine, tyrosine, and/or tryptophan), and phosphorylation (i.e., the addition of a phosphate group to serine, threonine, tyrosine, and/or histidine). For a recent review on the post-translational modification of proteins produced in eukaryotes, see Mowen and David, “Unconventional post-translational modifications in immunological signaling,” 15(6) Nat Immunol 512-20 (2014); and Blixt and Westerlind, “Arraying the post-translational glycoproteome (PTG),” 18 Curr Opin Chem Biol. 62-9 (2014).

Immunoglobulins (a.k.a. “antibodies”) are examples of proteins having multiple polypeptide chains and extensive post-translational modifications. The canonical immunoglobulin protein (e.g., IgG) comprises four polypeptide chains-two light chains and two heavy chains. Each light chain is linked to one heavy chain via a cystine disulfide bond, and the two heavy chains are bound to each other via two cystine disulfide bonds. Immunoglobulins produced in mammalian systems are also glycosylated at various residues (e.g., at asparagine residues) with various polysaccharides, and can differ from species to species, which may affect antigenicity for therapeutic antibodies (see Butler and Spearman, “The choice of mammalian cell host and possibilities for glycosylation engineering”, 30 Curr Opin Biotech 107-112 (2014)).

Antibodies are often used as therapeutic biomolecules. The term “antibody”, as used herein, includes immunoglobulin molecules comprised of four polypeptide chains, two heavy (H) chains and two light (L) chains interconnected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10-9 M, at least 10-1 M; at least 10-11 M; or at least 10-12 M, as measured by surface plasmon resonance, e.g., BIACORE™ or solution-affinity ELISA.

The phrase “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The antibody mixtures characterized herein are subjected to stress. The stress may be selected from thermal stress, photo stress, low pH stress, agitation stress, sheer stress or combinations thereof, for example. In a particular embodiment, the stress is thermal stress. Thermal stress conditions can include the antibody mixture stored or incubated at 30 °C, 35 °C, 40 °C, 41 °C, 42 °C, 43 °C, 44 °C, 45 °C, 46 °C, 47 °C, 48 °C, or 50 °C. The antibody mixture can be under thermal stress conditions for an amount of time, such as but not limited to 1 hour, 2 hours, 4 hours, 8 hours, 16 hours, 24 hours, 48 hours, 72 hours, 1 week, 2 weeks, 1 month, 2 months, 3 months, 4 months, 5 months, 6 months, or more. For example, the antibody mixture can be under thermal stress conditions at 40 °C for 1 month or 3 months. Another example, the antibody mixture can be under thermal stress conditions at 45 °C for 1 month or 3 months.

### EXAMPLES

The inventions are further described by the following Examples, which do not limit the inventions in any manner and are applicable to all sections of the descriptions of the inventions and the aspects of the inventions. The order of performance of the below Examples can be altered or combined as determined by the person of skill in the art in view of the teachings and data contained herein.

### Example 1: Methods

Chemicals and reagents. mAb-1 (hIgG4), mAb-2 (hIgG4), and their corresponding antigens are all produced at Regeneron (Tarrytown, NY). Deionized water was provided by a Milli-Q integral water purification system installed with a MilliPak Express 20 filter (Millipore Sigma, Burlington, MA). Formic Acid (FA, 98-100% Suprapur for trace metal, 1.11670) was purchased from Millipore Sigma. Ammonium acetate (MS grade, 73594-100G-F), urea (U5128-500G), and iodoacetamide (IAA; A955-4) were obtained from Sigma-Aldrich (St. Louis, MO). Acetonitrile (ACN; liquid chromatography-MS grade, A955-4), dithiothreitol (DTT, A39255), trifluoroacetic acid (TFA, 28904), tris(2-carboxyethyl)phosphine (TCEP; Thermo Scientific, 20491) and Invitrogen UltraPure 1 M Tris-HCl buffer, pH 8.0 (15568-025) were purchased from Thermo Fisher Scientific (Waltham, MA). Glacial acetic acid (0714-2.5L) was purchased from VWR (Radnor, Pa). 2-propanol (IPA, 34965-4X4L) was purchased from Honeywell (Charlotte, NC). Sequencing grade modified trypsin (V5111) was purchased from Promega (Madison, WI). PNGase F (P0704L) was purchased from New England Biolabs (Ipswich, Ma).

Native SEC-MS analysis with PCD. Co-formulated samples were diluted using Tris-HCl, pH 7.5. Samples were deglycosylated using PNGase F (1 U/5 μg, 45 C, 1 hr, 100 mM Tris-HCl final). Samples were injected onto either a ACQUITY UPLC Protein BEH (200 Å, 1.7 μm, 4.6 mm×150 mm) or a ACQUITY UPLC Protein BEH (200 Å, 1.7 μm, 4.6 mm×300 mm) column using an isocratic gradient of 150 mM ammonium acetate for 5 minutes (0.6 mL/min) or 20 minutes (0.2 mL/min), respectively. If applicable, PCD solution (60% ACN, 4% FA) was applied via a post-column splitting T at an equal flow rate (1:1 mixing). Isopropanol was used to desolvate. The flow was then split into a microflow (<10 μL/min) for nanoelectrospray ionization (nESI)-MS detection. The remaining flow was used for UV detection (280 nm). A Thermo Q-Exactive UHMR (Thermo Fisher Scientific, Bremen, Germany) equipped with a Microflow-Nanospray Electrospray Ionization (MnESI) Source and a Microfabricated Monolithic Multinozzle (M3) emitter (Newomics, Berkeley, CA) was used for MS analysis. A detailed experimental setup and instrument parameters can be found in previous publications [25, 33].

Subunit nSEC-MS with PCD analysis. Stressed material was generated in house by the Formulation Development Group. The Fc domain was removed using FabRICATOR (1 U/ug, 37 C, 1 hr, 50 mM Tris-HCl) to generate the F (ab)′2 and Fc fragments. Samples utilizing both PNGase F and FabRICATOR were incubated at 37 C for 1 hr in 50 mM Tris-HCl. The mAbs interchain disulfide bonds were partially reduced using DTT (5 mM final, 30 minutes, 37 C) and alkylated (20 mM final, 25 C, 30 minutes, dark). Samples were separated and analyzed as described above.

Middle-down subunit analysis. Samples were prepared and separated as described above. A Thermo Fisher Orbitrap Eclipse Tribrid (Thermo Fisher Scientific, Bremen, Germany) with a HESI source was utilized for analysis. The HCD collision energy was set to 20 for MS2 fragmentation.

Data Analysis. The acquired intact MS and subunit MS data were processed and deconvoluted with PMI Intact Mass (Protein Metrics, Cupertino, CA). Mass ranges varied depending on the species of interest. Other parameters are as follows: charge vector spacing 0.2; baseline radius 2; and charge range 5-200. Default settings were used for the other parameters.

Byonic (Protein Metrics, Cupertino, CA) was used to search the acquired reduced peptide mapping data. Cleavage sites were set to KR with one missed cleavage. The precursor mass tolerance was 5 ppm, while the fragment mass tolerance was 20 ppm. Common post-translational modifications, including Met/Trp oxidation, Asn deamidation, Asn ammonia loss and Asp dehydration, were set as common variables. Up to 2 common modifications were allowed.

Middle-down results were processed using ProSight Native (Proteinaceous, Evanston, IL). The scan range was averaged across all MS2 scans. Default settings were used for analysis. Fragments of interest were manually validated.

Structural Analysis and Modeling. VL/VH structures for mAb-1 and mAb-2 were obtained from in-house resources. mAb-1 VL/mAb-2 VH crossover structural model was built via superposing mAb-1 VL to mAb-2 VL. Total interface area, hydrophobic interface area and shape complementarity between VL and VH were calculated for mAb-1, mAb-2 and mAb-1/mAb-2.

‘Triangular’ heterocomplex model was built by superimposing mAb-1 VL to mAb-2 VL. Manual adjustment of conformation was performed for VH-FW4 of mAb-1 and CH1 of mAb-2 followed by local energy minimization to allow for proper geometry of the affected residues. All structural analysis and modeling were performed in MOE [43].

Reduced Peptide Mapping. mAb 1 was subjected to reduced peptide mapping, including reduction, alkylation and enzymatic digestion. The mAb (5 mg/mL final) was diluted to 40 μL using 5 mM acetic acid. Reduction was achieved by adding TCEP-HCl (4.76 mM final) followed by heating at 80 °C for 10 minutes. After cooling to room temperature, urea (3M final) in Tris-HCl buffer was introduced to facilitate protein denaturation. The sample was then alkylated with iodoacetamide (2 mM final) and digested with trypsin (1:20 w/w). The digestion process was conducted at 30 °C for 4 hours. The reaction was completed by acidifying with TFA (0.2% final), vortexing and centrifuging.

A Q-Exactive Quadrupole-Orbitrap MS (Thermo Fisher Scientific, San Jose, CA) coupled to a Waters I-Class HPLC (Waters, Milford, MA) was used for peptide mapping analysis. The purified peptides were analyzed using a BEH C18 column (ACQUITY UPLC Peptide BEH C18 Column 130 Å, 1.7 μm, Waters, Milford, MA). Separation was achieved using a programmed gradient starting at 0.1% Mobile Phase B (0.045% TFA in ACN) with a fixed flow rate of 250 nL/minute. The gradient was maintained at 99.9% Mobile Phase A (0.05% TFA in Water) for the initial 5 minutes. Mobile Phase A was then decreased to 60% over the next 85 minutes, followed by a further decrease to 10% within 5 minutes. This condition was maintained for 11 minutes to ensure complete elution of all analytes. Post-equilibration was performed by returning to 99.9% Mobile Phase A for 19 minutes before the next injection. The column was incubated at 40 °C.

### Example 2: Detection of a Truncated Heterodimer Species in a Two-mAb Cocktail by SEC-PCD-MS

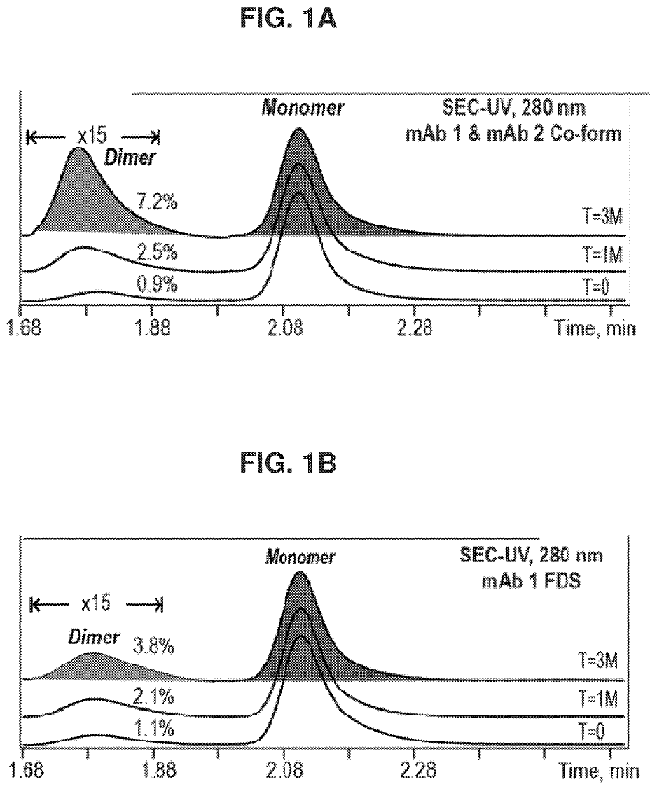

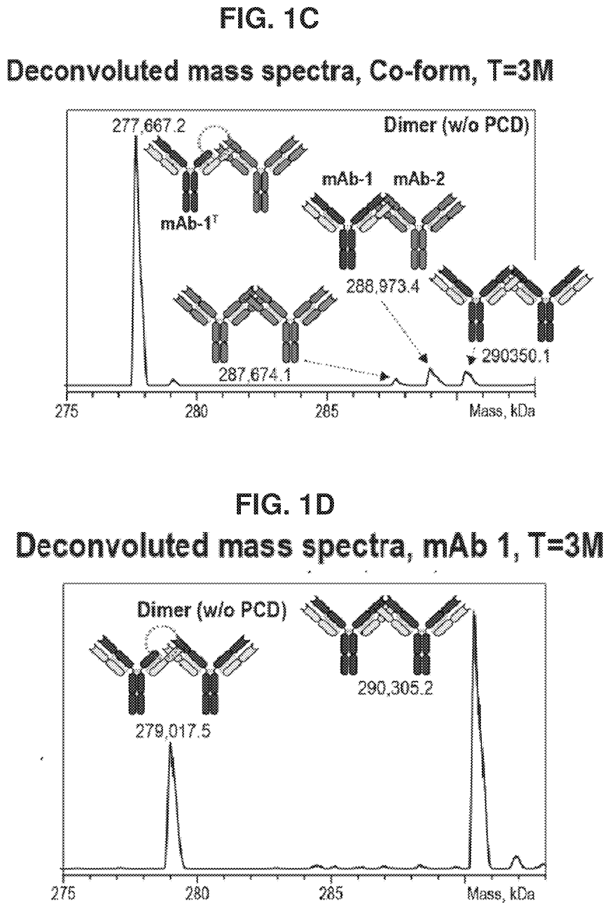

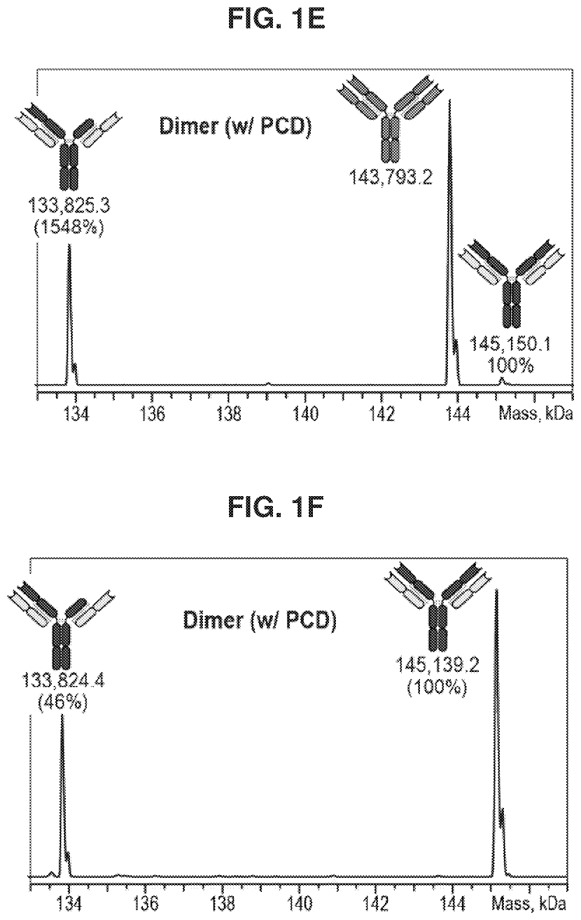

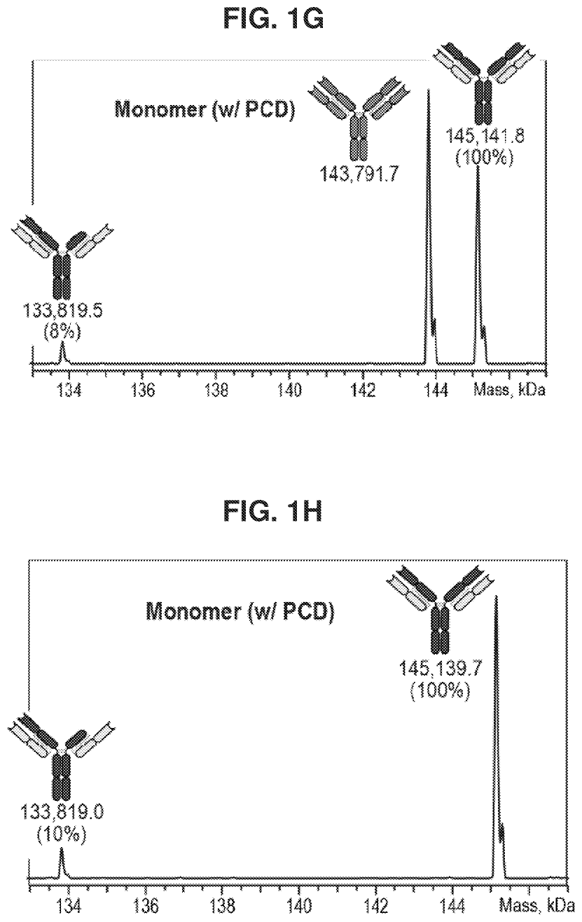

Thorough characterization of HMW species is crucial during the development of co-formulated mAb cocktails, as it offers valuable insights into aggregation mechanisms and establishes a foundation for risk assessment. Utilizing SEC-UV, the propensity for HMW formation under thermal stress (40 °C) was compared between a co-formulated mAb cocktail (mAb-1+mAb-2) and its individually formulated counterpart (mAb-1). Interestingly, in the co-formulated sample, the relative abundance of the dimer increases at a much faster rate over three months compared to the individually formulated mAb-1 sample (FIGS. 1A-1B). While these results may suggest a preferential hetero-interaction between mAb-1 and mAb-2 over mAb-1 self-interaction, concrete evidence was lacking. To ascertain which interactions were driving dimer growth, nSEC-MS was employed. Examination of the deconvoluted mass spectra of the dimer region revealed the presence of an unconventional HMW species in both the co-formulated and the single-mAb samples, which exhibited masses (~280 kDa) slightly smaller than the expected full-length dimers (~290 kDa, including mAb-1 homodimer, mAb-2 homodimer, and mAb-1/mAb-2 heterodimer) (FIGS. 1C-1D). In addition, the abundance of this unusual HMW species was much higher in the co-formulated sample than in the mAb-1 only sample. As these presumably truncated dimer species were also observed in the mAb-1 only sample, they were likely associated with truncations in mAb-1. However, detailed characterization was still necessary to elucidate the exact composition of these HMW species. To this end, SEC-PCD-MS is a highly effective tool for in-depth characterization of mAb HMW species [25]. By applying PCD solvent after native SEC separation, this technique enabled online dissociation of HMW species into their constituent components before MS detection. Using this technique, the predominant components contributing to the HMW formation in the stressed, co-formulated sample were identified as truncated mAb-1 (mAb-1T) and full-length mAb-2 (FIG. 1E). In contrast, the primary components responsible for the HMW formation in the stressed, individually formulated mAb-1 sample were identified as mAb-1T and full-length mAb-1 (FIG. 1F).

Interestingly, it is also found that the mAb-1T species (133 kDa) was significantly enriched in the dimer region compared to the monomer region in both the co-formulated and the single-mAb samples. Using the full length mAb-1 as a comparator, the relative abundance of the mAb-1T was enriched from 8% in the monomer region to ~1548% in the dimer region in the stressed, co-formulated sample, representing a ~195-fold enrichment (FIGS. 1E and 1G). In contrast, the relative abundance of the mAb-1T increased from 10% in the monomer region to ~46% in the dimer region in the stressed, mAb-1 only sample (FIGS. 1F and 1H), representing a ~5-fold enrichment. These results indicated that the mAb-1T exhibited a greater propensity for dimerization compared to the full-length mAb-1. Most intriguingly, this species also showed a clear preference for forming heterodimers with mAb-2 over self-interacting with mAb-1. This preference explained the faster dimer growth in the co-formulated sample compared to the individually formulated mAb-1 under identical thermal stress conditions.

### Example 3: The Clipped VH Domain Remains Associated Prior to Dimerization, Forming Native-Like mAb Structure

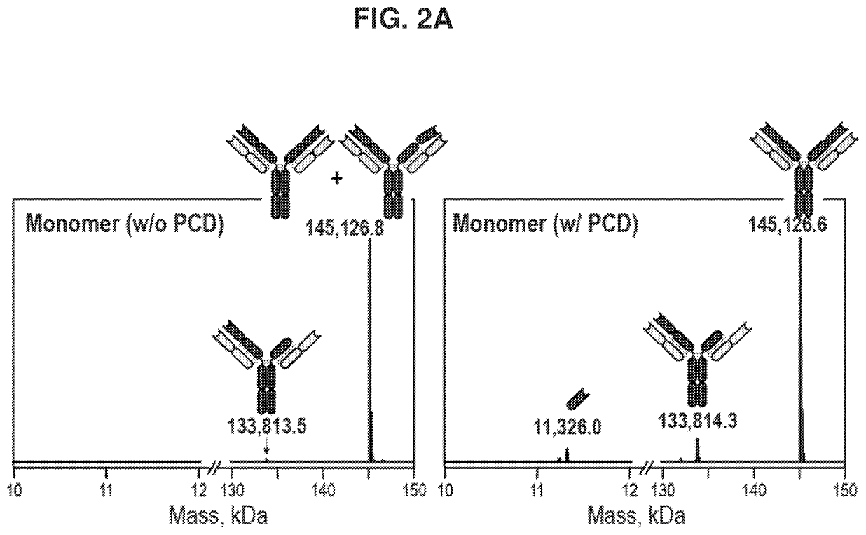

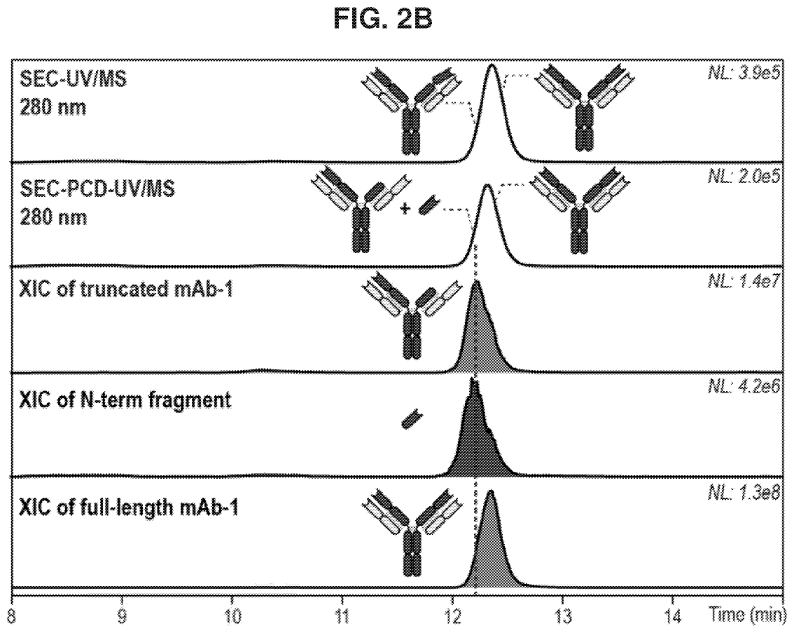

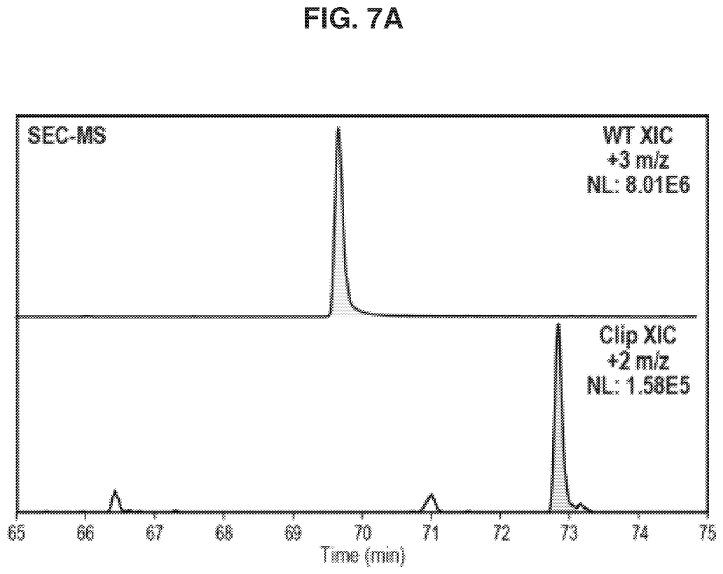

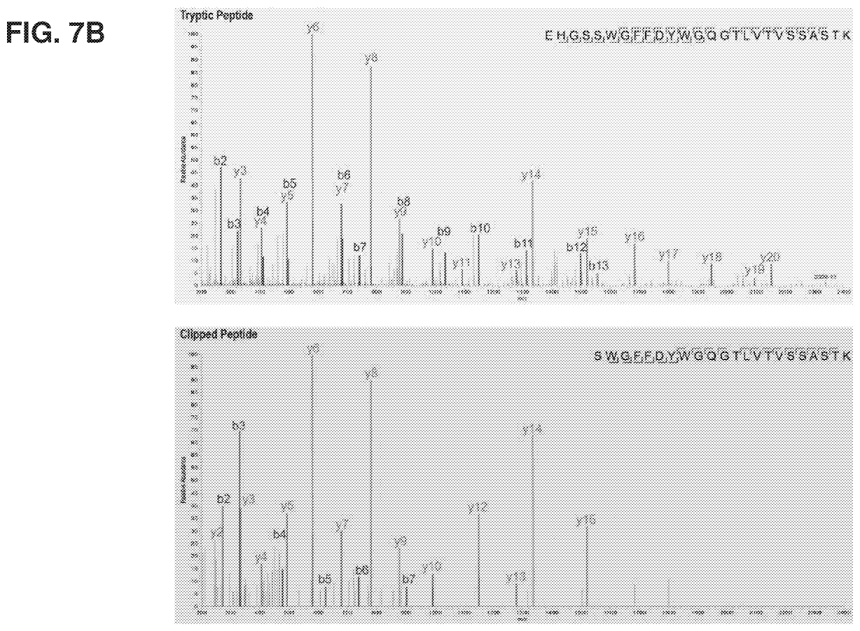

To identify the truncation site within the mAb-1 molecule, SEC-PCD-MS analysis of the stressed, individually formulated mAb-1 sample was further performed at a higher MS resolution setting (12.5 k vs 6.25 k). In the absence of PCD, analysis of the monomer region primarily revealed the full-length mAb-1, while a very low abundance of the mAb-1T species was also observed (FIG. 2A, left panel). In contrast, with PCD enabled, the analysis clearly revealed an increased level of the mAb-1T species, as well as the emergence of its complementary fragment (11,326 Da) within the same spectrum (FIG. 2A, right panel). By matching with the cDNA-predicted sequence, the truncation site was localized to the N-terminal of Ser103 within the heavy chain (HC) CDR3, which was subsequently confirmed by peptide mapping analysis (FIG. 2A; FIGS. 7A-7B). It was hypothesized that, after clipping, this HC variable region fragment (VH, ~11 kDa) likely continued to interact with the remaining mAb molecule (i.e., mAb-1T, 133 kDa) through strong VH-VL non-covalent interaction, forming a native-like mAb structure. This hypothesis can be effectively demonstrated by comparing the elution profiles of each fragment using the extracted ion chromatograms (XICs) from the SEC-PCD-MS analysis. As shown in FIG. 2B, the XICs of the VH fragment (~11 kDa) and the mAb-1T species (~133 kDa) exhibited nearly identical elution times and profiles, despite their distinct size differences. This indicates that the VH fragment and the mAb-1T species were associated during SEC separation and were only dissociated under PCD-MS conditions. In addition, this analysis also revealed that this clipped, mAb-like species eluted slightly earlier than the unmodified mAb during SEC separation. This observation is consistent with several recent reports, highlighting that CDR3-clipped mAbs often elute slightly before the SEC main peak [34, 35]. The authors proposed that the early shift of the retention time of the clipped species was likely caused by weaker secondary interactions with SEC matrix. Furthermore, under native MS detection conditions, the clipping event should theoretically result in an 18 Da mass increase due to the hydrolysis of the amide bond, which involves the addition of one water molecule. This shift, however, would be very difficult to observe due to the low abundance of this clipped species and its co-elution with the main mAb. Nevertheless, an examination of the deconvoluted mass spectra across the main peak (from nSEC-MS data) did highlight a slight mass increase in the leading edge of the peak (data not shown), indicating the presence of the clipped species. These results highlighted the effectiveness of the SEC-PCD-MS approach in identifying low levels of protein backbone clippings in mAb samples.

Finally, since only two fragments were identified, the clipping in mAb-1 appeared to be highly specific under thermal stress conditions. Indeed, protein backbone fragmentation occurring N-terminal to serine (Xaa-Ser) is not unusual and is well-documented in literature [36], which is proposed to occur via a two-step mechanism. First, the neighboring N-terminal peptide bond undergoes nucleophilic attack from the serine alcohol, forming an oxazolidine intermediate. This intermediate then rearranges to an ester intermediate that is hydrolyzed by water to complete peptide-bond cleavage [37]. Due to the high specificity observed within mAb-1, the local microenvironment (e.g., solvent accessibility, pKa, etc.) likely plays a pivotal role in cleavage propensity.

### Example 4: Identification of Domains Involved in Heterodimerization

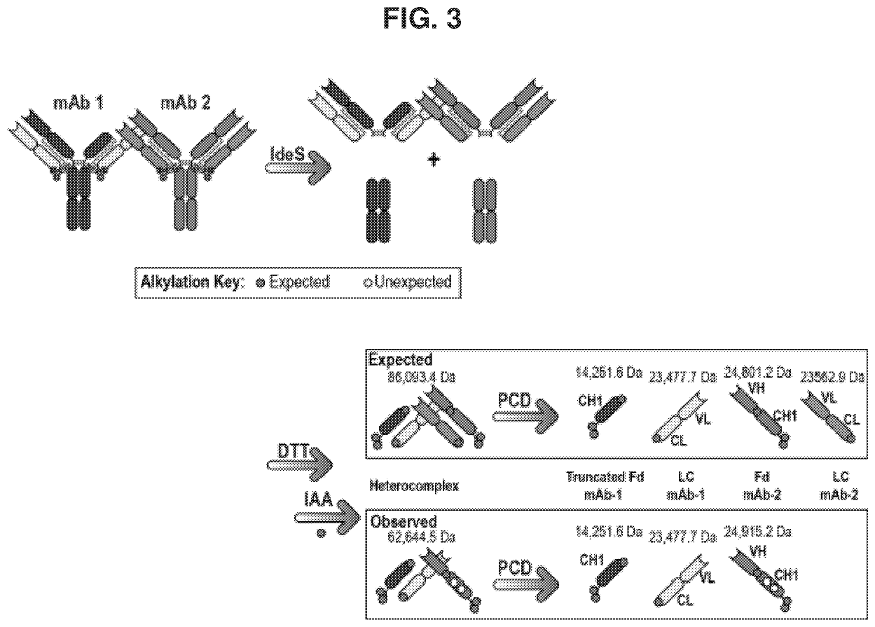

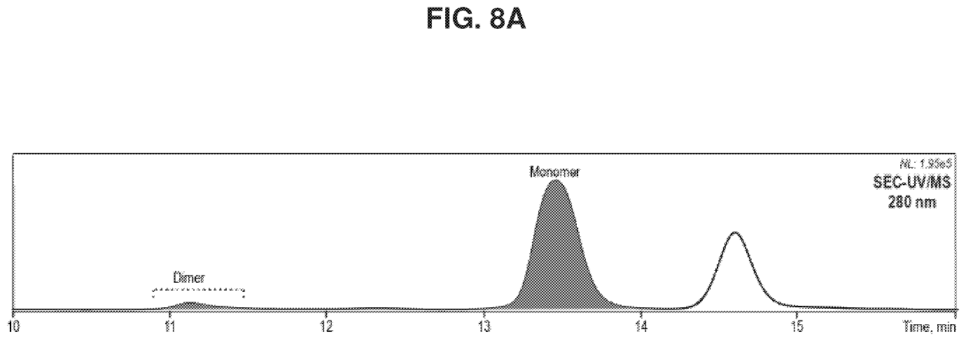

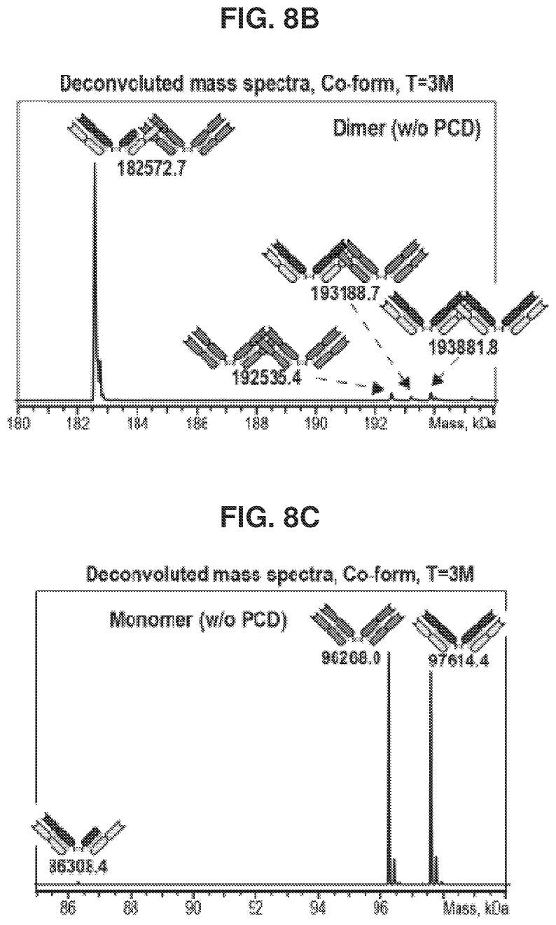

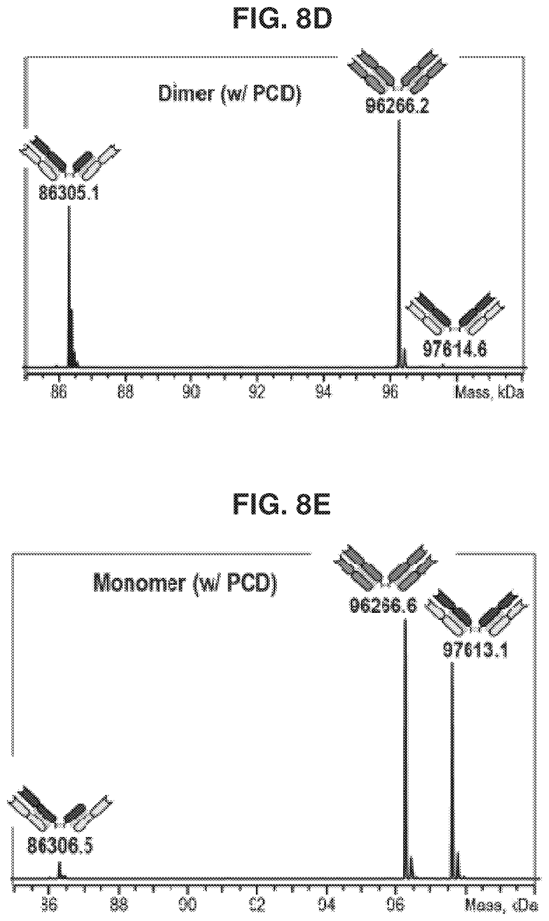

While previous analyses have clearly shown a preference for heterodimerization between the CDR3-clipped mAb-1 molecule (mAb-1T) and the full-length mAb-2 molecule, the underlying biochemical root cause remains unidentified. To ascertain the domains involved in the hetero-dimerization, several strategies were systematically employed to deconstruct the interaction. IdeS digestion was first employed on the stressed, co-formulated mAb sample to localize the interaction to either the fragment crystallizable region (Fc) or the Fab2 arms (FIG. 3). After IdeS treatment, the sample was subjected to SEC-PCD-MS analysis. In the absence of PCD, a truncated Fab2 heterodimer species (M.W., ~183 kDa) was observed as the major HMW species in the dimer region (FIGS. 8A and 8B). Subsequent PCD-MS analysis confirmed that the truncated Fab2 dimer was composed of the truncated Fab2 from mAb-1 and the intact Fab2 from mAb-2, confirming a Fab-Fab interaction (FIG. 8D). Similar to the observations at the intact mAb level, enrichment of the truncated mAb-1 Fab2 species was evidence in the dimer region compared to the monomer region (FIGS. 8C and 8E). However, it remained unclear which domains were involved in heterodimerization.

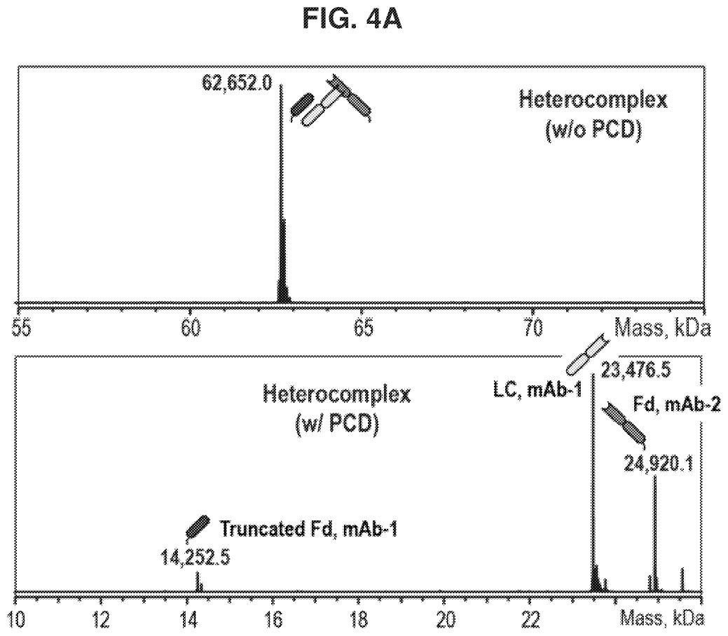

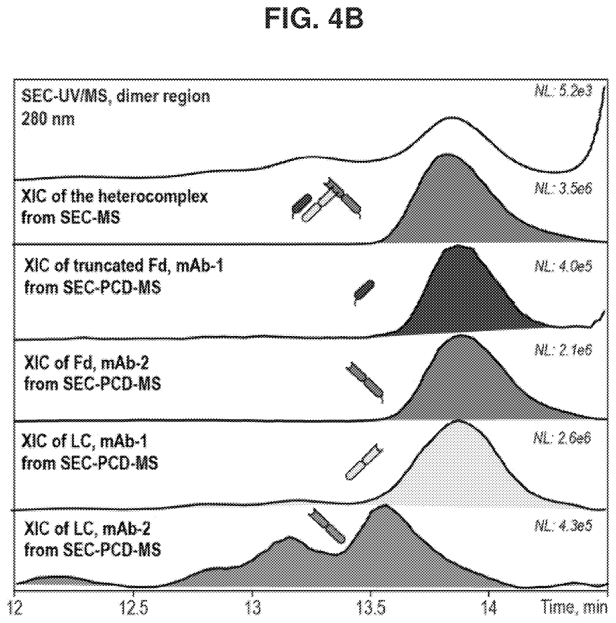

To improve resolution, IdeS-treated sample was further subjected to partial reduction under native conditions to selectively reduce the inter-chain disulfide bonds and further localize the interaction to the constituent subunits (FIG. 3). The newly formed free thiols (1 from LC and 3 from Fd (i.e., VH and CH1 domains)) were subsequently capped by iodoacetamide (IAA)-based alkylation reaction to prevent re-formation of disulfide bonds. Importantly, within a properly folded mAb molecule, the intra-domain disulfide bonds are highly resistant to reduction under native conditions due to their poor solvent accessibility [38, 39]. This feature could also be utilized to probe the potential structural changes of domains involved in dimerization. Following the further treatment, nSEC-MS analysis, both with and without PCD treatment, revealed a non-covalent heterocomplex (~63 kDa) that was comprised of the truncated Fd (FdT) from mAb-1, LC from mAb-1, and Fd from mAb-2 (FIGS. 4A-4B). Interestingly, in contrast to the expected Fab-Fab heterodimer, LC from mAb-2 was absent from the observed heterocomplex (FIGS. 3, 4A-4B). To ensure this was not caused by gas phase dissociation of mAb-2 LC from the non-covalent complex, the elution profile of each component was compared based on the XICs from the SEC-PCD-MS analysis. This comparison clearly demonstrated that the FdT from mAb-1, LC from mAb-1, and Fd from mAb-2 all exhibited nearly identical elution profiles, which also closely matched the elution profile of the heterocomplex without PCD (albeit minor retention time shift due to the PCD flow) (FIG. 4B). In contrast, the XIC of mAb-2 LC displayed a markedly different elution profile, indicating its absence from the heterocomplex in the solution phase.

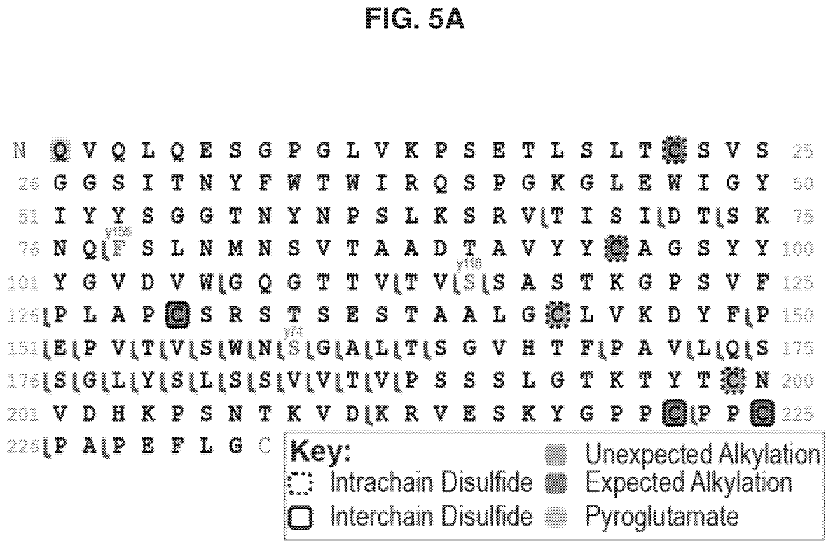

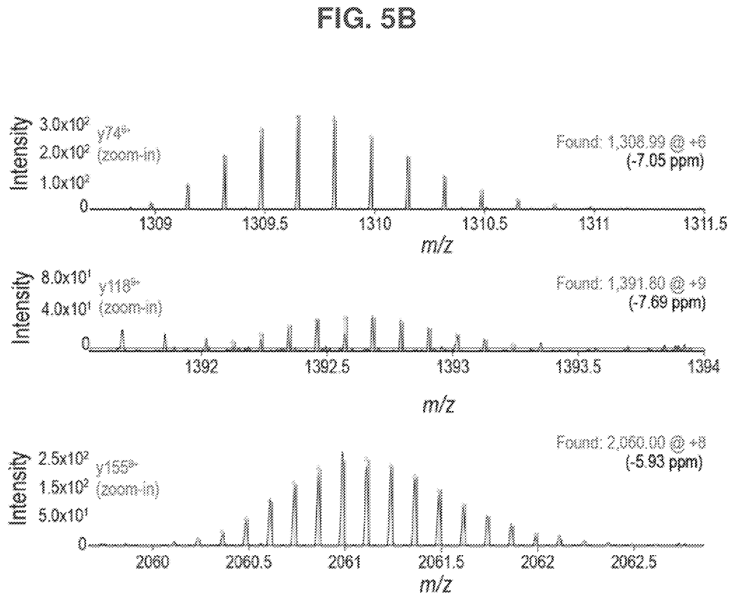

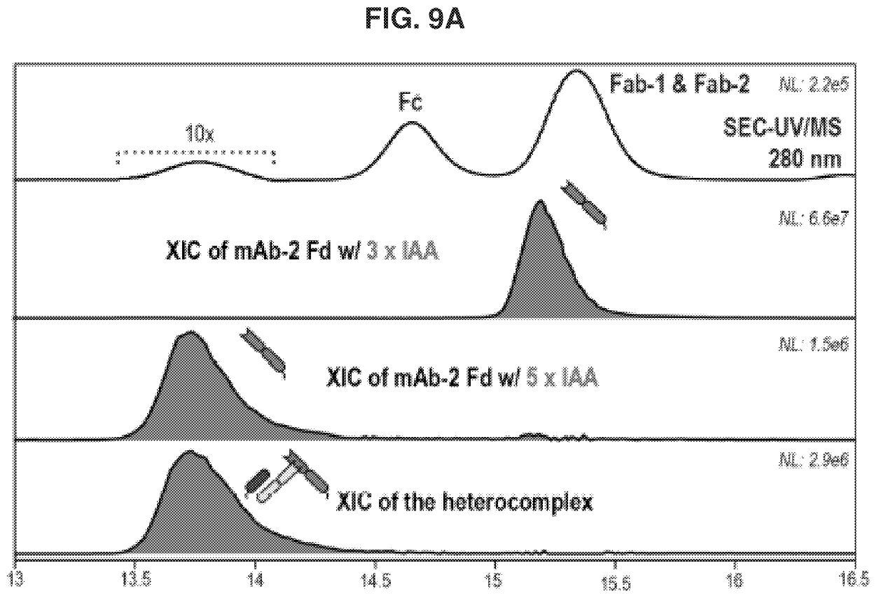

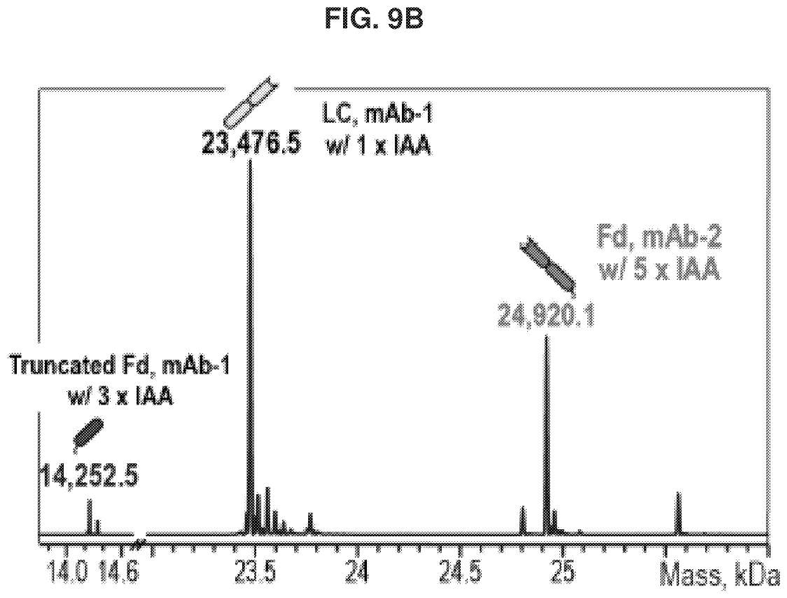

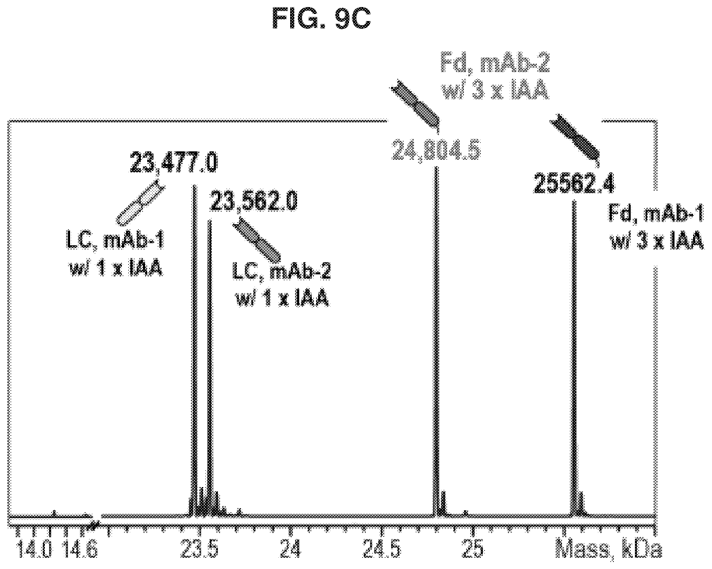

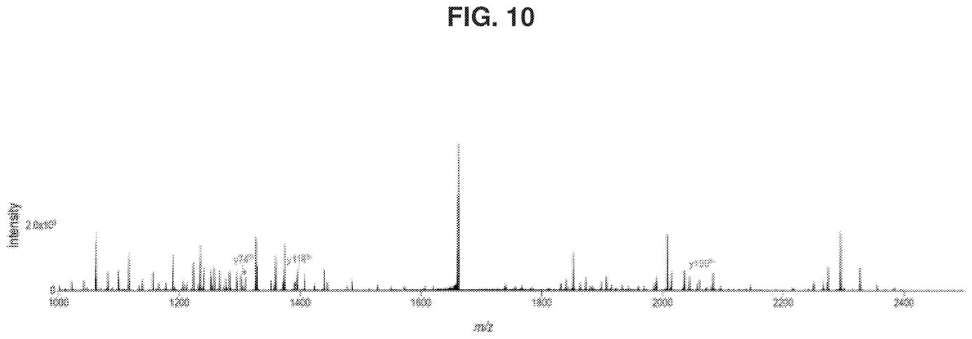

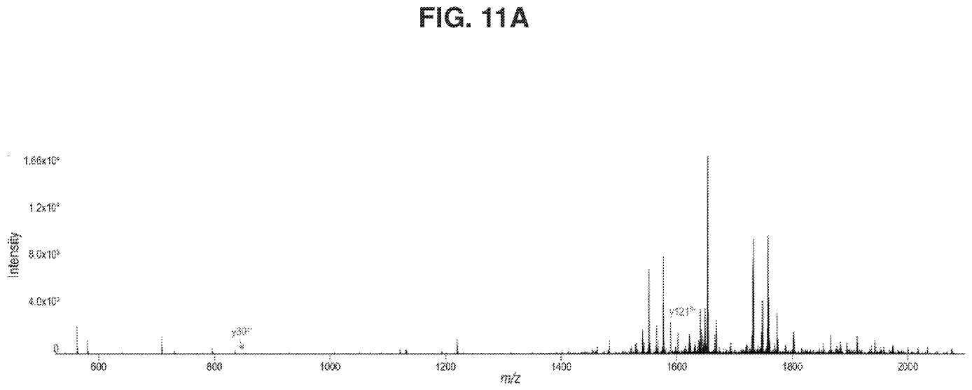

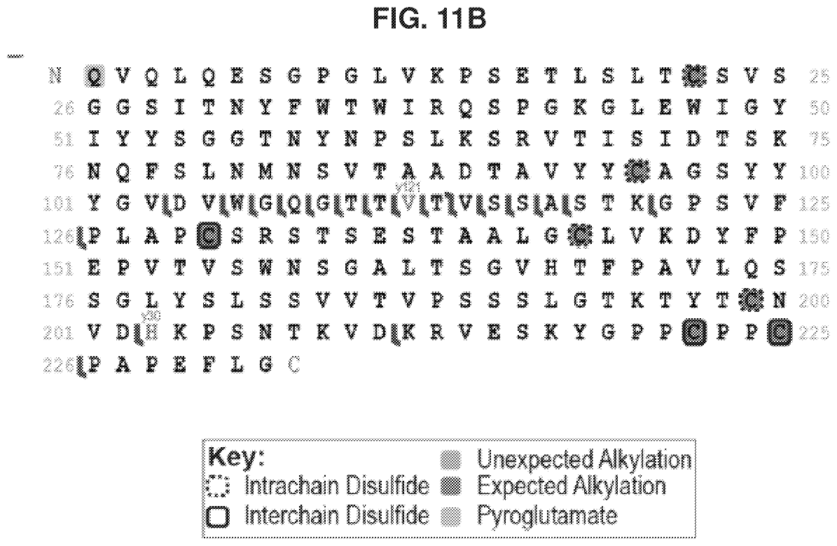

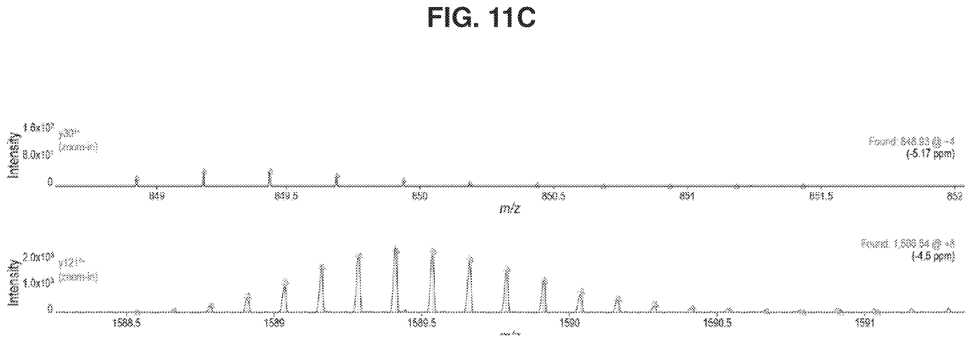

Upon close examination of the deconvoluted mass of the heterocomplex, a mass deviation of ~+114 Da was observed (expected: 62,530.5 Da, observed: 62,652.0 Da) (FIG. 3 and FIG. 4A). PCD-MS analysis successfully localized the mass deviation to the Fd of mAb-2 (expected mass with 3×IAA addition: 24,804.5 Da, observed mass: 24,920.1 Da) (FIG. 3 and FIG. 4A). Interestingly, based on the XIC analysis, this mass addition was not observed on the mAb-2 Fd from the Fab fragment, which exhibited the expected number of IAA modification (FIGS. 9A-9C). Given that Cys alkylation by IAA introduces a mass increase of approximately +57 Da per site, it was hypothesized that an intra-domain disulfide bond within mAb-2 Fd from the hetero-complex was unexpectedly reduced and subsequently alkylated. To confirm this hypothesis, middle-down MS analysis using higher-energy collisional dissociation (HCD) was conducted on the mAb-2 Fd fragment with ~114 Da mass addition. Rich HCD fragmentation was observed from an originally disulfide-bonded region within the CH1 domain, suggesting the reduction of this disulfide bond in this species (FIG. 5A and FIG. 10). Further analysis of the b- and y-ions also localized the additional two IAA modifications to Cys147 and Cys203 residues within the mAb-2 CH1 domain (FIG. 5B). In contrast, fragmentation of the unmodified species (i.e., mAb-2 Fd with 3×IAA addition) in the Fab region resulted in no fragmentation within the CH1 disulfide-bonded region (FIGS. 11A-11C). This observation aligns well with the fact that the HCD fragmentation is highly inefficient at generating b and y ions within the disulfide-bonded regions of IgG subunits, as it requires the concurrent cleavage of both the peptide amide bond and the disulfide bond [39-41]. These data confirmed the unexpected reduction (and alkylation) of mAb-2 CH1 domain disulfide bond in the heterocomplex under the limited reduction conditions. This significantly increased susceptibility of this originally well-buried disulfide bond may be explained by its increased solvent accessibility, due to the absence of the cognate CH1-CL pairing caused by the lack of mAb-2 LC in the heterocomplex. Interestingly, because the mAb-2 VH domain disulfide bond did not exhibit a similar susceptibility increase, it is plausible that it remained shielded from reduction by interacting with the mAb-1 VL domain, suggesting a VH (truncated)-VL domain interaction within the heterocomplex (FIG. 3). Together, these findings indicate that the truncated heterodimer, formed specifically between the CDR3-clipped mAb-1 and the full-length mAb-2, are largely maintained through the mAb-1 VL and mAb-2 VH interaction, which is driven by a specific mAb-1 CDR clipping event.

### Example 5: Structural Analysis and Modeling Support a Stable Complex Formed via mAb-1 LC and mAb-2 HC

Structural analysis and modeling were performed to elucidate the biophysical underpinnings of the truncated mAb-1 VL interacting with the VH of the full-length mAb-2, resulting in two potential complementary mechanisms. Using a structural model based on mAb-1 VL/mAb-2 VH, the VL/VH interface area, hydrophobic interface area, and shape complementarity were calculated to compare the relative strength between different VL/VH interactions (Table 1). Analysis of properly paired chains, specifically mAb-1 Fd with mAb-1 LC and mAb-2 Fd with mAb-2 LC, reveals similar shape complementarity (i.e., the degree to which the interface contour boundaries of VL and VH align with each other) among VL/VH interfaces (0.38-0.43) (Table 1). However, mAb-2 exhibits a greater hydrophobic surface area of 670 Å² compared to 530 Å² for mAb-1. Minor differences in total surface area also favor mAb-2 interactions (1795.1 A2) over mAb-1 (1758.4 A2). These findings suggest stronger interactions between VL and VH in mAb-2. Interestingly, the mAb-1 VL/mAb-2 VH interface have both larger buried total surface (1814.3 A2) and hydrophobic surface (650 A2) areas than properly paired mAb-1 chains, indicating a stronger interaction for mAb-2 VH than mAb-1 VH towards mAb-1 VL binding (Table 1). This clearly explains the preference of mAb-1 VL for mAb-2 VH over its own cleaved cognate mAb-1 VH in the presence of mAb-2.

TABLE 1
Structural analysis on interface features among
native mAb-1, mAb-2 and mAb-1/mAb-2 VL/VH pairs.

| | mAb-1 VL | mAb-2 VL | mAb-1 VL |
|---|---|---|---|
| Interface Features | mAb-1 VH | mAb-2 VH | mAb-2 VH |
| Total VL/VH interface | 1758.4 | 1795.1 | 1814.3 |
| area (Å²) | | | |
| Hydrophobic interface | 530 | 670 | 650 |
| area (Å²) | | | |
| Shape | 0.38 | 0.43 | 0.42 |
| Complementarity | | | |

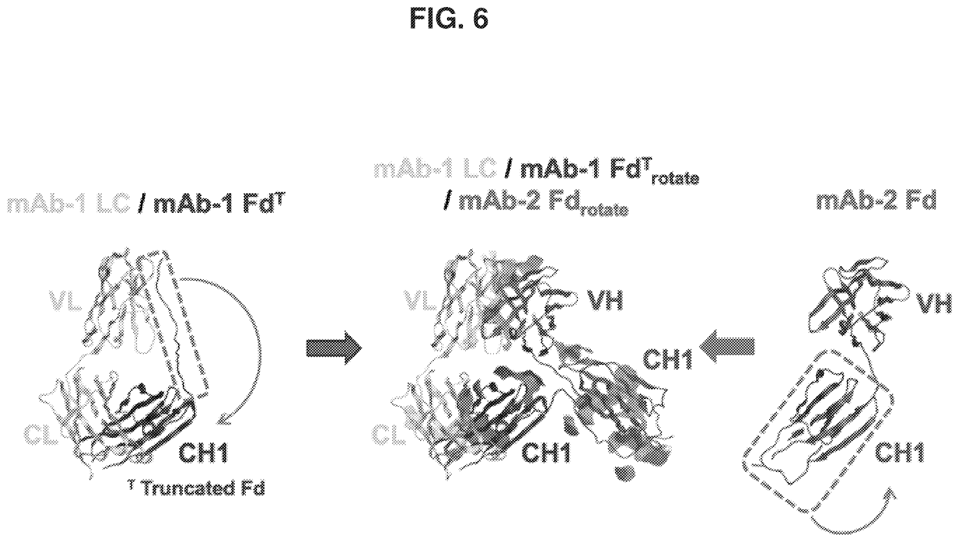

In addition to quantitative data regarding the interface and surface area measurements, the possibility of qualitative data impacting heterodimer formation was explored, focusing particularly on the structural characteristics that influence the preference of mAb-1 VL for mAb-2 VH. Analysis of the available structures of mAb-1 and mAb-2 revealed that the VH-truncated mAb-1 Fd (mAb-1 FdT) contains a long framework 4 (FW4) chain preceding the CH1 domain (FIG. 6). It is known that the VH-CH1 interaction can adopt various conformations and elbow angles [42]. Consequently, both the heavy chain framework 4 (HC-FW4) of mAb-1 and the CH1 domain of mAb-2 can exhibit rotational flexibility around the linker region connecting the VH and CH1 domains (FIG. 6, left). Conformational flexibility can expose new hydrophobic regions on both the FW4 of mAb-1 and the CH1 of mAb-2, facilitating their association and leading to the stabilization of a non-covalently linked heterocomplex (mAb-1 FdT, mAb-1 LC, and mAb-2 Fd). Further, the proposed structural model also illustrates that FW4 has incomplete coverage of the whole CL-CH1 interface in mAb-2 CH1 domain (FIG. 6), corroborating the increased reduction susceptibility of intra-CH1 disulfide bond because of partial exposure of this domain in the heterocomplex (FIGS. 5 and 6). In summary, the ‘triangular’ heterocomplex model underscores the significance of both quantitative and qualitative data in supporting the dimerization mechanism between the VL of the truncated antibody with the VH of another antibody molecule.

### Discussion

Heterodimers represent a novel impurity and may serve as critical quality attributes in co-formulated therapeutic antibody products. A multi-faceted MS approach was used to identify heterodimer formation and dissect the underlying dimerization mechanisms. These findings suggest that the heterodimerization interaction between the CDR3-clipped mAb-1 and the full length mAb-2 is characterized by a dimerization mechanism, likely dependent on conformational changes. While the effects of CDR3-clipping on mAbs have been extensively studied concerning potency, hydrophobicity, thermal stability and other properties, our work is the first to demonstrate the formation of CDR3-clipped heterodimers in FDCs [34, 35]. The implications of this truncated heterodimer on safety, particularly regarding immunogenicity and potency, remain to be fully elucidated.

Given the potential for CDR3-clipped heterodimers to influence both the safety and efficacy of therapeutic antibody products, further investigation is essential. Future studies should prioritize characterizing the immunogenic properties of these heterodimers and their impact on therapeutic potency. Additionally, developing predictive models for heterodimer formation could significantly reduce development costs and enhance patient safety by identifying problematic formulations early in the process.

Overall, the results of this study provide valuable insights into the molecular interactions and structural stability of co-formulated mAbs, which are crucial for the development of effective therapeutics. By exploring the dimerization mechanisms and detailing the specific interactions between mAb domains, this research enhances our understanding of mAb behavior, specifically within FDCs. These findings will inform the design and optimization of future FDCs, improving the efficacy and safety of co-formulated mAbs in clinical applications.

### CITATIONS

1. Kaplon, H., et al., Antibodies to watch in 2022. MAbs, 2022. 14(1): p. 2014296.
2. Mullard, A., FDA approves 100th monoclonal antibody product. Nat Rev Drug Discov, 2021. 20(7): p. 491-495.
3. Doud, M. B., J. M. Lee, and J. D. Bloom, How single mutations affect viral escape from broad and narrow antibodies to H1 influenza hemagglutinin. Nat Commun, 2018. 9(1): p. 1386.
4. Coronavirus (COVID-19) Update: FDA Revokes Emergency Use Authorization for Monoclonal Antibody Bamlanivimab. 2021: www.fda.gov/news-events/press-announcements/coronavirus-covid-19-update-fda-revokes-emergency-use-authorization-monoclonal-antibody-bamlanivimab.
5. Zahavi, D. and L. Weiner, Monoclonal Antibodies in Cancer Therapy. Antibodies (Basel), 2020. 9(3).
6. Litman, G. W., J. P. Rast, and S. D. Fugmann, The origins of vertebrate adaptive immunity. Nat Rev Immunol, 2010. 10(8): p. 543-53.
7. Bonilla, F. A. O., H. C., Adaptive immunity. Journal of Allergy and Clinical Immunology, 2010. 125(2): p. S33-S40.
8. Schmidt, C., The benefits of immunotherpy combinations. 2017.
9. Wei, J., et al., Current landscape and future directions of bispecific antibodies in cancer immunotherapy. Front Immunol, 2022. 13: p. 1035276.
10. Krieg, D., G. Winter, and H. L. Svilenov, It is Never Too Late for a Cocktail-Development and Analytical Characterization of Fixed-dose Antibody Combinations. J Pharm Sci, 2022. 111(8): p. 2149-2157.
11. Gao, J. J., et al., FDA Approval Summary: Pertuzumab, Trastuzumab, and Hyaluronidase-zzxf Injection for Subcutaneous Use in Patients with HER2-positive Breast Cancer. Clin Cancer Res, 2021. 27(8): p. 2126-2129.
12. FDA Approves Breast Cancer Treatment That Can Be Administered At Home By Health Care Professional. 2020.
13. FDA Approves First Treatment for Ebola Virus. 2020.
14. FDA approves Opdualag for unresectable or metastatic melanoma. 2022.
15. Chauhan, V. M., et al., Advancements in the co-formulation of biologic therapeutics. J Control Release, 2020. 327: p. 397-405.
16. Kim, J., et al., Analytical characterization of coformulated antibodies as combination therapy. MAbs, 2020. 12(1): p. 1738691.
17. Cao, M., et al., Charge variants characterization and release assay development for co-formulated antibodies as a combination therapy. MAbs, 2019. 11(3): p. 489-499.
18. Mueller, C., U. Altenburger, and S. Mohl, Challenges for the pharmaceutical technical development of protein coformulations. J Pharm Pharmacol, 2018. 70(5): p. 666-674.
19. Kuhne, F., et al., Identification of Hetero-aggregates in Antibody Co-formulations by Multi-dimensional Liquid Chromatography Coupled to Mass Spectrometry. Anal Chem, 2023. 95(4): p. 2203-2212.
20. Jin, X., et al., Charge Variants Characterization of Co-Formulated Antibodies by Three-Dimensional Liquid Chromatography-Mass Spectrometry. Biomolecules, 2024. 14(8).
21. Candreva, J., et al., Dual-detection approach for a charge variant analysis of monoclonal antibody combination products using imaged capillary isoelectric focusing. Electrophoresis, 2022. 43(16-17): p. 1701-1709.
22. Xu, C. K., S.; Pierson, N. A.; Quiroz, J.; Kochert, B.; Yang, X.; Wylie, D.; Strulson, C. A., Development, validation, and implementation of a robust and quality control-friendly focused peptide mapping method for monitoring oxidation of co-formulated monoclonal antibodies. Analytical and Bioanalytical Chemistry 2022. 414: p. 8317-8330.
23. Paul, R. G.-M., A.; Stahlberg, H.; Lauer, Me. E.; Rufer, A. C.; Beck, H.; Briguet, A.; Schnaible, V.; Buckel, T.; Boeckle, S., Structure and Function of Purified Monoclonal Antibody Dimers Induced by Different Stress Conditions. Pharmaceutical Research, 2012. 29: p. 2047-2059.
24. Sauna, Z. E., et al., Evaluating and Mitigating the Immunogenicity of Therapeutic Proteins. Trends Biotechnol, 2018. 36(10): p. 1068-1084.
25. Yan, Y., et al., Post-Column Denaturation-Assisted Native Size-Exclusion Chromatography-Mass Spectrometry for Rapid and In-Depth Characterization of High Molecular Weight Variants in Therapeutic Monoclonal Antibodies. J Am Soc Mass Spectrom, 2021. 32(12): p. 2885-2894.
26. Lebowitz, J., M. S. Lewis, and P. Schuck, Modern analytical ultracentrifugation in protein science: a tutorial review. Protein Sci, 2002. 11(9): p. 2067-79.
27. Hughes, H., et al., A multi-tiered analytical approach for the analysis and quantitation of high-molecular-weight aggregates in a recombinant therapeutic glycoprotein. AAPS J, 2009. 11(2): p. 335-41.
28. Lowe, D. D., K; Rouet, R; Schofield, P.; Jermutus, L.; Christ, D., Aggregation, stability, and formulation of human antibody therapeutics. Advances in Protein Chemistry and Structural Biology, 2011. 84: p. 41-61.
29. Zolls, S., et al., Particles in therapeutic protein formulations, Part 1: overview of analytical methods. J Pharm Sci, 2012. 101(3): p. 914-35.
30. Boyd, D., et al., Isolation and characterization of a monoclonal antibody containing an extra heavy-light chain Fab arm. MAbs, 2018. 10(3): p. 346-353.
31. Lazar, A. C., et al., Analysis of the composition of immunoconjugates using size-exclusion chromatography coupled to mass spectrometry. Rapid Commun Mass Spectrom, 2005. 19(13): p. 1806-14.
32. Muneeruddin, K., et al., Characterization of small protein aggregates and oligomers using size exclusion chromatography with online detection by native electrospray ionization mass spectrometry. Anal Chem, 2014. 86(21): p. 10692-9.
33. Yan, Y., et al., Versatile, Sensitive, and Robust Native LC-MS Platform for Intact Mass Analysis of Protein Drugs. J Am Soc Mass Spectrom, 2020. 31(10): p. 2171-2179.
34. Atsumi, Y., et al., Identification and Characterization of a Monoclonal Antibody Variant Species with a Clipping in the Complementarity Determining Region Isolated by Size Exclusion Chromatography Under Native Conditions. J Pharm Sci, 2021. 110(10): p. 3367-3374.
35. Atsumi, Y., et al., Clip Formation in the Complementarity Determining Region of Bevacizumab Lowers Monomer Stability and Affinity for Both FcRn and FcgammaR: A Comprehensive Characterization of the Clipped Variant Including its Higher Order Structure. J Pharm Sci, 2022. 111(12): p. 3243-3250.
36. Li, W., et al., Identification and characterization of monoclonal antibody fragments cleaved at the complementarity determining region using orthogonal analytical methods. J Chromatogr B Analyt Technol Biomed Life Sci, 2017. 1048: p. 121-129.
37. Vlasak, J. and R. Ionescu, Fragmentation of monoclonal antibodies. MAbs, 2011. 3(3): p. 253-63.
38. Liu, H., et al., Ranking the Susceptibility of Disulfide Bonds in Human IgG1 Antibodies by Reduction, Differential Alkylation, and LC-MS Analysis. Analytical Chemistry, 2010. 82(12): p. 5219-5226.
39. Huang, X., et al., Development of a Novel Label-Free Subunit HILIC-MS Method for Domain-Specific Free Thiol Identification and Quantitation in Therapeutic Monoclonal Antibodies. J Am Soc Mass Spectrom, 2024. 35(12): p. 3019-3027.
40. Fornelli, L., et al., Structural Analysis of Monoclonal Antibodies with Top-down and Middle-down Electron Transfer Dissociation Mass Spectrometry: The First Decade. Chimia (Aarau), 2022. 76(1-2): p. 114-126.
41. Rush, M. J. P., et al., Top-Down Characterization of Proteins with Intact Disulfide Bonds Using Activated-Ion Electron Transfer Dissociation. Anal Chem, 2018. 90(15): p. 8946-8953.
42. McConnell, S. A. and A. Casadevall, New insights into antibody structure with implications for specificity, variable region restriction and isotype choice. Nat Rev Immunol, 2025.
43. ULC, C. C. G., Molecular Operating Environment (MOE). 2025(2024.0601).

1. A method of identifying a dimerization region in a first antibody in an antibody mixture, wherein the method comprises the steps of
(a) subjecting the antibody mixture to stress;
(b) conducting native size-exclusion chromatography (nSEC) on the stressed antibody mixture from step (a);
(c) applying post-column denaturation (PCD) solvent after nSEC separation, thereby enabling dissociation of high-molecular weight (HMW) species into their monomeric constituent components; and
(d) analyzing the PCD solvent treated components by mass spectrometry (MS), thereby identifying the dimerization region in the first antibody in the antibody mixture.

2. The method according to claim 1, wherein the antibody mixture of step (a) is subjected to thermal stress.

3. The method according to claim 1, wherein the antibody mixture of step (a) is heated to a temperature greater than 40 °C.

4. (canceled)

5. The method of claim 1, wherein the antibody mixture of step (a) is thermally stressed for about 1 hour to about 6 months.

6. (canceled)

7. The method of claim 1, wherein the MS analysis of step (d) further compares HMW species from the antibody mixture and HWM species from a single-antibody sample of the first antibody.

8. (canceled)

9. The method of claim 1, wherein the PCD treatment reveals an increased level of a truncated antibody species and its complementary fragment.

10. The method of claim 9, wherein the truncated antibody species are truncated at a complementarity-determining region (CDR).

11. The method of claim 10, wherein the truncated antibody species comprises a domain opposite the truncated CDR.

12. The method of claim 11, wherein the domain opposite the truncated CDR is the dimerization region.

13. The method of claim 1, wherein the antibody mixture comprises the first antibody, a second antibody, and optionally one or more additional antibodies.

14. The method of claim 13, wherein an intact Fab₂ arm of the second antibody dimerizes with the dimerization region in the first antibody.

15. The method of claim 1, wherein the antibody mixture comprises monoclonal antibodies.

16. The method of claim 1, wherein the antibody mixture is in Tris-HCl buffer having a pH ranging from 7.0 to 8.5.

17. The method of claim 1, further comprising digesting the antibody mixture with IdeS prior to nSEC in step (b) to separate a fragment crystallizable region (Fc) and a Fab₂ arms.

18. The method of claim 17, wherein the IdeS-treated antibody mixture is subjected to steps (b)-(d).

19. The method of claim 17, wherein the subsequent PCD-MS analysis identifies that the dimerization region comprises a truncated Fab₂ from the first antibody and an intact Fab₂ is from the second antibody, thereby identifying the dimerization region comprise a Fab-Fab interaction.

20. (canceled)

21. A system for characterizing a dimerization region in an antibody mixture comprising a first antibody, wherein the system comprises a native size-exclusion chromatography (nSEC), a post-column denaturation (PCD) solvent, and mass spectrometry (MS) (SEC-PCD-MS) to analyze the antibody mixture to characterize at least one multimeric antibody form comprising one or more truncated antibody species and at least one multimeric antibody form comprising one or more full length antibody species in one or more elution peaks.

22. The system according to claim 21, wherein the multimeric antibody form is selected from the group consisting of a dimer, trimer, tetramer, pentamer, hexamer, and heptamer forms.

23. The system according to claim 22, wherein the antibody mixture is subjected to thermal stress.

24. The system according to claim 21, wherein the SEC-PCD-MS can generate extracted ion chromatograms to elucidate the truncated antibody species.


SEQUENCE LISTING (3 sequences)

SEQ ID NO: 1 - protein, 232 aa, synthetic construct
QVQLQESGPGLVKPSETLSLTCSVSGGSITNYFWTWIRQSPGKGLEWIGYIYYSGGTNYNPSLKSRVTISIDTSKNQFSLNMNSVTAADTAVYYCAGSYYYGVDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLG

SEQ ID NO: 2 - protein, 26 aa, synthetic construct
EHGSSWGFFDYWGQGTLVTVSSASTK

SEQ ID NO: 3 - protein, 22 aa, synthetic construct
SWGFFDYWGQGTLVTVSSASTK